(19) United States
(12) Patent Application Publication
(10) Pub. No.: US 2021/0371036 A1
(43) Pub. Date: Dec. 2, 2021

(54) ELECTRIC VEHICLE WITH ONE OR MORE UVC LIGHT SOURCES

(71) Applicant: Acton, Inc., Livermore, CA (US)

(72) Inventors: Peter Treadway, Livermore, CA (US); Andrew Tam, Livermore, CA (US)

(21) Appl. No.: 17/331,892

(22) Filed: May 27, 2021

Related U.S. Application Data
(60) Provisional application No. 63/030,923, filed on May 28, 2020.

(51) Int. Cl. B62J 45/00; B62J 6/00; B62K 3/00; A61L 2/10

(52) U.S. Cl. CPC B62J 45/00; B62J 6/00; B62K 3/002; A61L 2202/16; B62K 2202/00; A61L 2202/11; A61L 2/10

(57) ABSTRACT

An electric vehicle includes a steering element with a handle bar element. The steering element has a hand grip portion, and at a distal end a plurality of UVC light sources producing light that is incident on the hand grip portion. The UVC light sources are coupled to a controller and a power source. A platform supports a rider of the electric vehicle. The electric vehicle includes: at least one front wheel and at least one rear wheel; an electric motor configured to provide a mechanical power to at least one of the front and rear wheels; and a battery housing configured to removably receive and hold one or more battery packs, wherein the one or more battery packs are configured to power the electric vehicle.

### BACKGROUND

### Field of the Invention

This invention relates generally to, and more particularly to electric vehicles with one or more UVC light sources.

### Description of the Related Art

Assorted lightning devices are provided as attachments appointed to be affixed to the external body of an electric vehicle and are not integrated within the electric vehicle. One vehicle light apparatus is removably mounted on a vehicle including a rectangular web of pliant material having opposed long sides with fasteners for wrapping around an elongate member of a vehicle. One type of vehicle includes a reversible reflective/fluorescent rectangular sleeve safety device having two strips of highly reflective material on one side with one half of a hook and loop fastening means and two strips of high-visibility fluorescent material on the opposite side with the remaining half of the hook and loop fastening device,

One type system includes a single light emitting diode (LED) light source operating with a plurality of fiber optic cables secured thereto to emit light through the holes, and an attaching surface. Various lighting devices are provided for implementation into devices appointed to be worn or operate in conjunction with a vehicle, such as safety helmets. These types of devices are not integrated within the frame of a vehicle, but are rather utilized in helmets or the like. As such, using these helmet lights provides a degree of safety, but lighting emitted from the frame of the vehicle would provide enhanced visibility of the operator even from further distances off.

Other lighting devices are constructed and utilized as indicator lights for vehicles, and do not operate or function to provide for sanitizing sources that a user is in contact with.

There is a need for improved electric vehicle with UVC light sources.

### SUMMARY

An object of the present invention is to provide improved electric vehicles.

A further object of the present invention is to provide electric vehicles with UVC light sources.

These and other objects of the present invention are achieved in an electric vehicle. The electric vehicle includes a steering element with a handle bar element. The steering element has a hand grip portion, and at a distal end a plurality of UVC light sources producing light that is incident on the hand grip portion. The UVC light sources are coupled to a controller and a power source. A platform supports a rider of the electric vehicle. The electric vehicle includes: at least one front wheel and at least one rear wheel; an electric motor configured to provide a mechanical power to at least one of the front and rear wheels; and a battery housing configured to removably receive and hold one or more battery packs, wherein the one or more battery packs are configured to power the electric vehicle.

### BRIEF DESCRIPTION OF THE DRAWINGS

FIG. 1 illustrates an electric powered vehicle according to embodiments of the disclosed technology.

FIG. 2 illustrates further detail of the electric powered vehicle of FIG. 1.

FIG. 3 illustrates further detail of the deck assembly and latch of FIGS. 1 and 2.

FIG. 4 illustrates detail of the scooter of FIGS. 1 and 2 with the latch in an open position.

FIG. 5 illustrates further detail of the scooter of FIGS. 1 and 2 with the latch in an open position.

FIG. 6 illustrates detail of the scooter of FIGS. 1 and 2 with the latch in an open position.

FIG. 7 illustrates further detail of the scooter of FIGS. 1 and 2 during installation, or removal, of the deck assembly.

FIG. 8 illustrates a quick twist electrical soft connector according to embodiments of the disclosed technology.

FIG. 9 illustrates a cushioned electrical connector according to embodiments of the disclosed technology.

FIG. 10 illustrates a compound locking assembly according to embodiments of the disclosed technology.

FIG. 11A illustrates a portion of the scooter during removal or installation of the deck assembly.

FIG. 11B illustrates the retention device for the loose portions of the electrical cables of the scooter.

FIG. 12 illustrates a hidden latch mechanism for the scooter.

FIG. 13 illustrates a process for a user to install a removable deck assembly into an electric powered vehicle according to embodiments of the disclosed technology.

FIG. 14 illustrates a process for a user to remove a removable deck assembly from an electric powered vehicle according to embodiments of the disclosed technology.

FIG. 15 illustrates one embodiment of electric vehicle with public and private keys, and vehicle to vehicle communication.

FIGS. 16-24 illustrates various embodiments of the present invention with UVC light sources.

FIG. 25 illustrates a block diagram of power and regeneration control logic in one embodiment of the present invention.

FIG. 26 is a block diagram showing communication between an electric vehicle and a mobile device in one embodiment of the present invention.

FIG. 27 is a partial view of an embodiment of the high visibility safety lighting system, wherein UVC source illumination segments are integrated within a handlebar assembly of an electric vehicle.

FIG. 28 is a schematic view of an electric vehicle with a plurality of UVC source illumination segments and transparent illumination segments integrated within the frame.

FIG. 29 is a circuit diagram of the high visibility safety lighting device wherein an optional acceleration sensor is utilized.

### DETAILED DESCRIPTION

Embodiments of the described technology provide electric powered vehicles having top-swappable batteries. The batteries may be attached to the underside of the deck of the scooter to form a removable deck assembly. The deck assembly may be removed from the top of the scooter by operating a latch and lifting a handle of the assembly. The deck assembly may be returned to the scooter in a similar manner.

In some embodiments, the battery may be electrically coupled to a motor of the scooter by electrical cables and an electrical connector. The electrical connector may be a quick twist connector that is opened and closed by twisting its halves in opposite directions.

In some embodiments, instead of using electrical cables, the scooter and deck assembly may include electrical connectors that mate when the deck assembly is installed in the scooter. The electrical connectors may be surrounded by cushions that protect the connectors from micro vibrations, dirt and water, and the like.

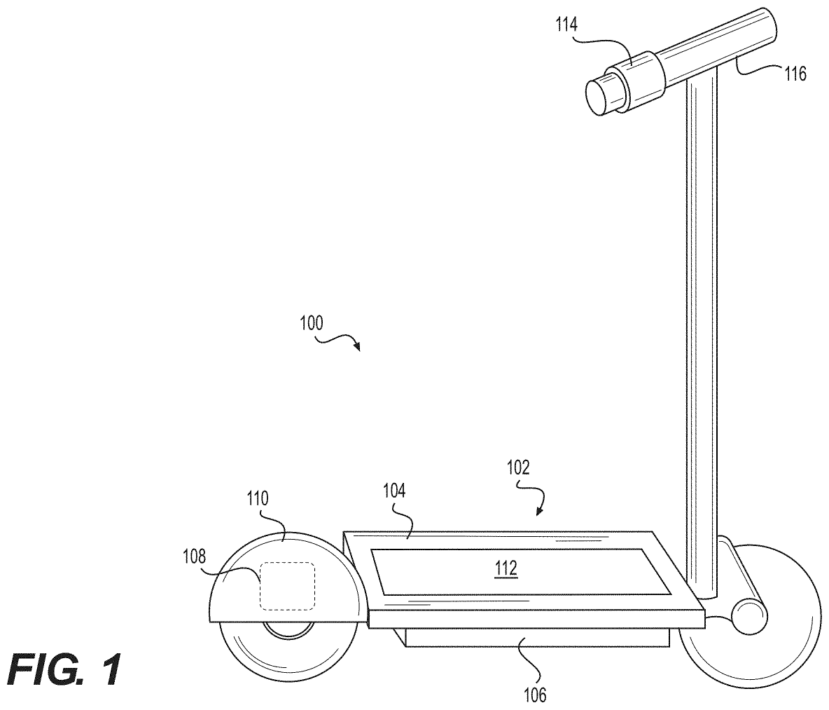

FIG. 1 illustrates an electric powered vehicle 100 according to embodiments of the disclosed technology. Referring to FIG. 1, the scooter 100 includes a deck assembly 102 removably attached to a frame 104 of the scooter 100. The deck assembly 102 includes a battery case 106 mounted underneath a deck 112. The battery case 106 includes one or more batteries (not shown). The batteries are electrically coupled to an electric drive motor 108, which is protected by a housing 110. The scooter 100 may be steered by turning a handlebar 116. The speed of the motor 108 may be controlled using a throttle 114 mounted on the handlebar 116.

The electric powered vehicle 100 is depicted in FIG. 1 as having only two wheels. However, it will be appreciated that the disclosed technology applies to scooters having any number of wheels. Furthermore, it will be appreciated that the disclosed technology applies to vehicles other than scooters, and having any number of wheels.

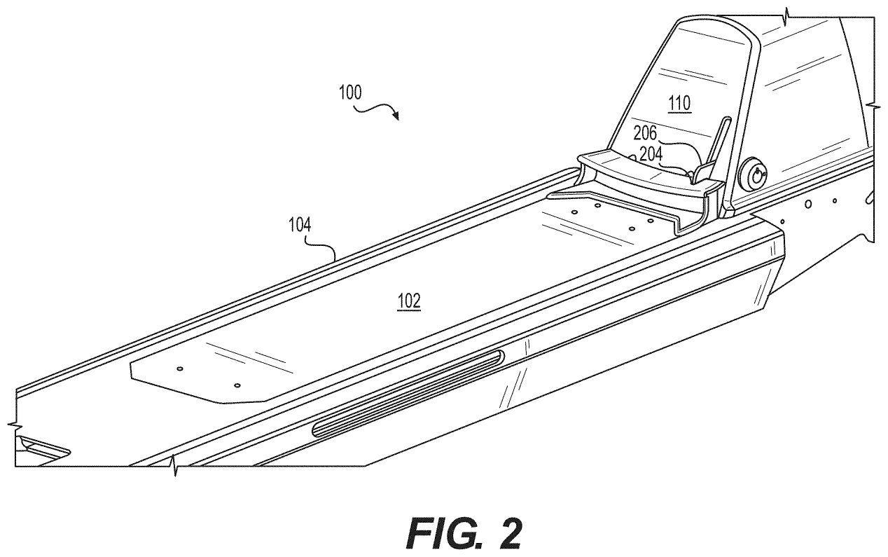

FIG. 2 illustrates further detail of the electric powered vehicle 100 of FIG. 1. Referring to FIG. 2, the removable deck assembly 102 is held flush with the frame 104 by a latch 206 when engaged in a notch 204. The latch 206 may be controlled by a latch mechanism (not shown) disposed within the housing 110.

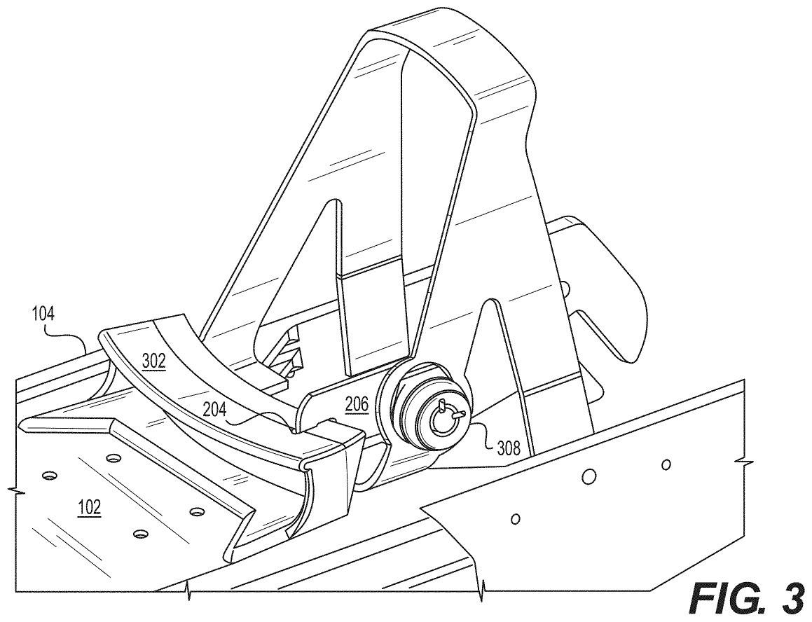

FIG. 3 illustrates further detail of the deck assembly 102 and latch 206 of FIGS. 1 and 2. Referring to FIG. 3, the deck assembly 102 may include a handle 302 to assist with the removal and installation of the deck assembly 102. The handle 302 may include a notch 204 to receive the latch 206. The deck assembly 102 may include a lock 308. The lock 308 may be operable to fix the latch 206 in an open position and/or a closed position, where the latch 206 secures the deck assembly 102 within the frame 104 when in the closed position. A key (not shown) may be inserted within lock assembly 308 to rotate the latch into, and out of, the notch 204, that is, between the closed position and an open position. When engaged with the notch 204, the latch retains the deck assembly 102 within the frame 104 of the scooter 100.

In the depicted embodiment, the lock assembly 308 is implemented as a physical lock, to be used with a physical key. But in other embodiments, the lock assembly 308 may be implemented in other ways. For example, the lock assembly 308 may be an electronic lock, which may be operated using an electronic key, fob, remote control, or the like. In embodiments where security is not required, the lock in the lock assembly 308 may be replaced with a knob, a button, or another mechanism. In any case, the lock assembly 308 may be hidden or disguised. This feature is especially useful in a ridesharing fleet, where users should not operate the lock assembly 308, or remove the deck assembly 102.

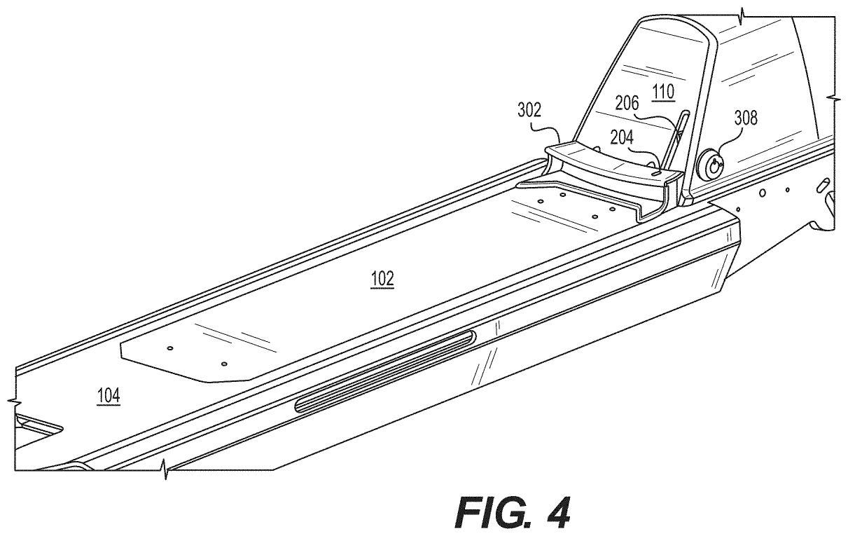

FIG. 4 illustrates detail of the scooter 100 of FIGS. 1 and 2 with the latch 206 in an open position. Referring to FIG. 4, the lock assembly 308 has been operated to rotate the latch 206 out of the notch 204. To protect the user from the latch, the latch 206 has been rotated to a position within the housing 110. The deck assembly 102 may now be removed from the scooter 100.

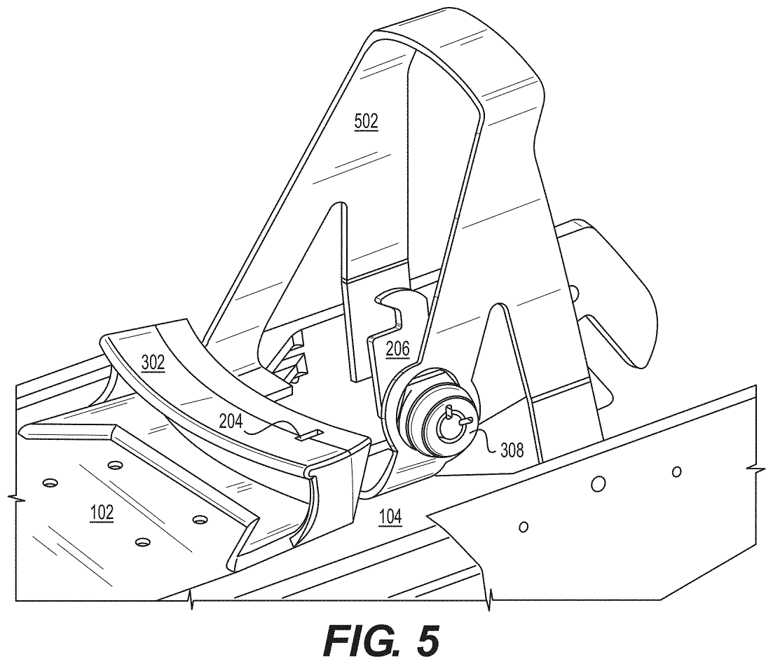

FIG. 5 illustrates further detail of the scooter 100 of FIGS. 1 and 2 with the latch 206 in an open position, and with the housing 110 removed. Referring to FIG. 5, the latch 206, and the lock assembly 308, are held in place by a strut 502 that is connected to the frame 104.

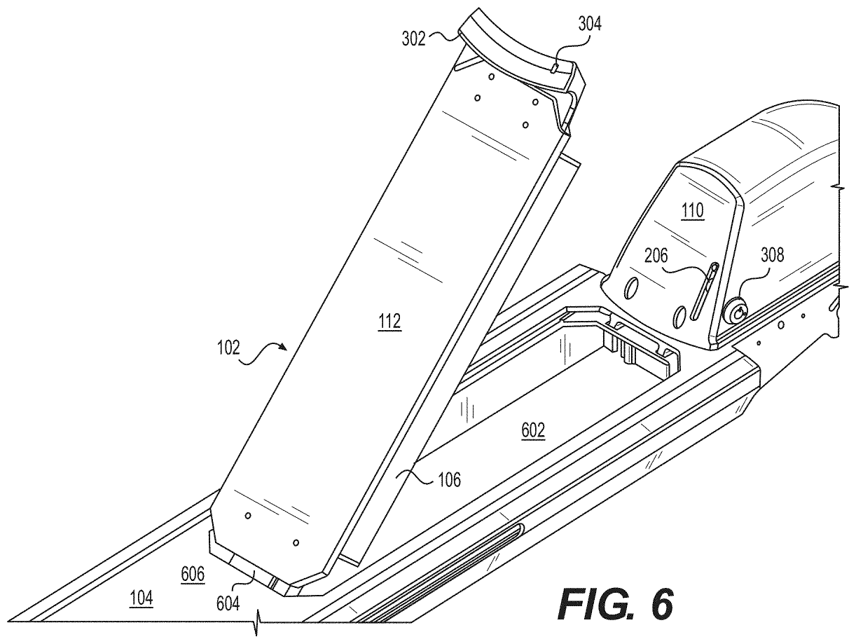

FIG. 6 illustrates detail of the scooter 100 of FIGS. 1 and 2 during installation, or removal, of the deck assembly 102. Referring to FIG. 6, the frame 104 has an upper surface 606, and an opening 602 in the frame 104 is visible. The opening 602 is formed so as to receive the deck assembly 102 when the deck assembly 102 is lowered into the opening of the frame from above the upper surface 606 of the frame. As can be seen in FIG. 6, the deck assembly 102 includes a protruding tongue 604 at the front of the deck assembly 102. During removal of the deck assembly 102, a user may lift the deck assembly 102 out of the opening of the frame 104 from above the upper surface 606 of the frame 104 by pivoting the deck assembly 102 upward about the tongue 604 using the handle 302, and then slide the deck assembly 102 slightly to the rear of the scooter 100 to disengage the tongue 604 from the frame 104. During installation of the deck assembly 102, a user may first insert the tongue 604 into the frame 104, pivot the deck assembly 102 downward into the opening 602 until flush with the frame 104, and then rotate the latch 206 into the notch 204 to secure the deck assembly 102 within the frame 104.

Also visible in FIG. 6 is the battery case 106. The battery case 106 may include one or more batteries (not shown), which may be cushioned with foam pads or similar materials. The battery case 106 may be integrated with the deck 112 to form the deck assembly 102, as noted above. The deck assembly 102 may be watertight to prevent damage to the batteries, and may be of automotive quality. This arrangement provides several advantages. In current designs, the battery case is mounted underneath a non-removable deck, for example using screws. In such designs, the batteries can only be removed by inverting the scooter, and unscrewing the battery case. During this process, the scooter may be damaged, the battery case may be damaged, and the screws may be lost. Furthermore, the user must have a tool such as a screwdriver. In contrast, in the described embodiments, the batteries may be removed without tools, by simply operating the latch 206 and lifting out the deck assembly 102. No tools are required. The scooter need not be inverted, and may remain on the ground, in a rack, or the like.

Other advantages are especially applicable to a fleet of shareable electric powered vehicles. In current fleets, the scooters are generally collected each evening, and taken to a charging facility where the batteries are charged. The charged scooters are then returned to scooter sharing locations the next morning. But in this arrangement, the scooters are unavailable for sharing while being charged. And this arrangement requires two trips per day: one trip to collect the scooters, and another trip to deploy them.

Embodiments of the disclosed technology solve both of these problems. With the disclosed removable deck assembly, the scooters need not be collected. Instead, only the deck assemblies may be collected. The scooters may be left in the sharing location, sharing racks, and the like. Furthermore, with a fleet of similar scooters, the deck assemblies are interchangeable. Therefore, an operator can replace a discharged battery pack with a fresh battery pack, requiring only one trip, and keeping the scooter available while the discharged battery pack is recharged. And because the disclosed deck assemblies are much smaller than the scooters, many more scooters can be serviced by a single truck than with current arrangements. In addition, because the disclosed deck assemblies weigh less than the scooter, there is less likelihood an operator will be injured while lifting them.

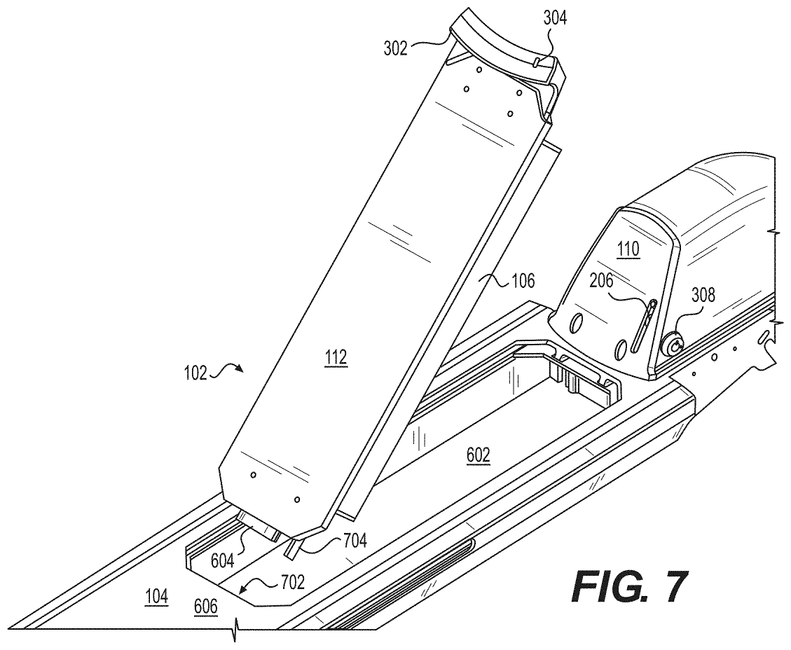

FIG. 7 illustrates further details of the scooter 100 of FIGS. 1 and 2 during installation, or removal, of the deck assembly 102 Referring to FIG. 7, the tongue 604 of the deck assembly 102 is free of the frame 104. As can be seen in FIG. 7, the frame 104 may feature a double-wall construction for rigidity and light weight. In the disclosed embodiment, a slot 702 may be formed between the walls of the frame 104 to receive the tongue 604. Also visible in the embodiment of FIG. 7 is a portion of an electrical power cable 704. The power cable 704 may provide power to the motor 108 of the scooter 100. To separate the deck assembly 102 from the scooter 100, the user may operate a connector of the power cable 704, as described in detail below.

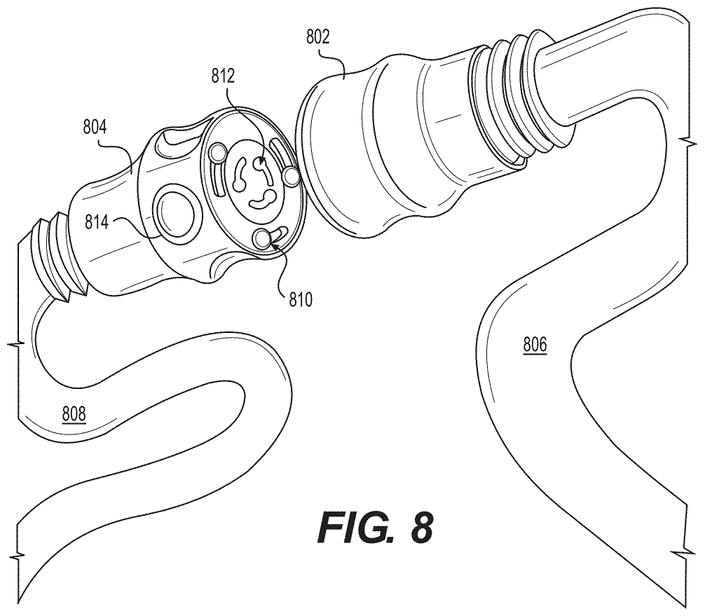

FIG. 8 illustrates a quick twist electrical soft connector according to embodiments of the disclosed technology. As used herein, the term “soft connector” is used to refer to a connector having two halves, where at least one of the halves is coupled to a flexible electrical cable. In some embodiments, the term “soft connector” is used to refer to a connector where both halves of the connector are coupled to respective flexible electrical cables. As described below, the flexible cable(s) serve to insulate the scooter from micro vibrations, a problem unique to vehicles such as scooters that have small, hard wheels. Referring to FIG. 8, the soft electrical connector includes a male half 802 and a female half 804. The halves 802, 804 are formed at the ends of electrical cables 806, 808, respectively. The illustrated soft connector is a quick twist connector that is opened and closed by twisting its halves 802, 804 in opposite directions. Accordingly, the female half 804 of the soft connector includes a plurality of curved slots 810, each including a round opening to receive a respective locking pin (not shown) of the male half 802. The electrical connectors may be implemented in a similar manner, as shown at 812.

In some embodiments, one half of the soft connector may include a locking indicator 814. The locking indicator 814 may shine red until the soft connector is completely closed, whereupon the indicator 814 may switch to green to indicate a positive lock of the soft connector.

One advantage of the disclosed quick twist electrical soft connector is that it mitigates the problem of micro vibrations. Vehicles such as automobiles and s are subject to vibrations caused by imperfections in the road surface. Vehicles with small, hard wheels, such scooters, are subject to these vibrations, and also to micro vibrations, which are caused by tiny imperfections in the road surface, for example such as the pebbles in a conglomerate road surface. Electrical connectors in particular are adversely affected by micro vibrations, which cause the mating electrical parts to rub together and thereby deteriorate. Gold plating on electrical connectors is particularly subject to this deterioration. In the disclosed embodiments, the lengths of electrical cables 806, 808 isolate the electrical connector from these micro vibrations, greatly reducing any wear the electrical connectors 812 experience.

Another advantage of the disclosed quick twist electrical soft connector is that it encourages users not to pull on the cables 806, 808 to open the soft connector. In conventional electrical connectors with no twist lock mechanism, users may be tempted to pull on the cables to open the connector. This abuse may shorten the life of the electrical cable and electrical connector considerably. But this is not possible with the twist connector. The user must grasp the soft connector halves in order to twist them in opposite directions. Consequently, the electrical soft connector and electrical cables 806, 808 may enjoy a longer lifespan.

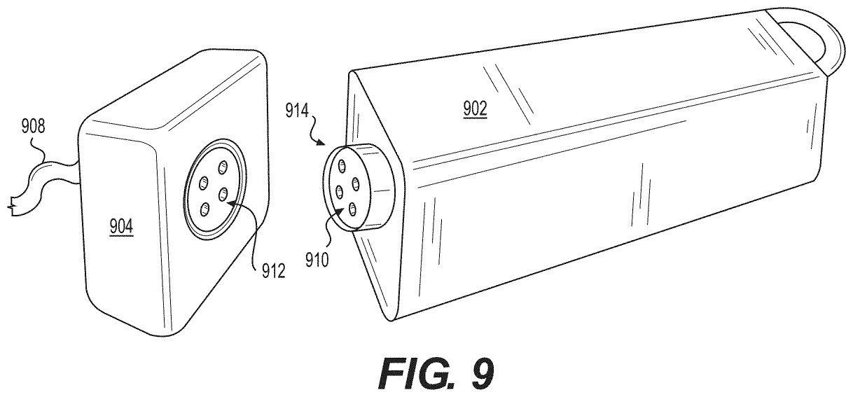

FIG. 9 illustrates a cushioned electrical connector according to embodiments of the disclosed technology. Referring to FIG. 9, a deck assembly 902 that includes a battery pack may be pressed against an elastic mounting block 904 during installation. The deck assembly 902, and the mounting block 904, include respective electrical connectors 910, 912 that are mated during installation of the deck assembly 902, thereby providing power from the battery pack to the motor through an electrical power cable 908. The mounting block 904 may be fabricated of an elastic material such as rubber to cushion the electrical connectors 910, 912 from micro vibrations. In the embodiment of FIG. 9, the elastic mounting block 904 is disposed upon the scooter.

But in other embodiments, an elastic mounting block may be disposed on the deck assembly 902 instead, or as well. For example, as shown in FIG. 9, the deck assembly 902 may include a second elastic mounting block 914 to further isolate the electrical connectors 910, 912 from micro vibrations. These elastic mounting blocks 904, 914 may also form a seal about the electrical connectors 910, 912 that protects the electrical connectors 910, 912 from water, dirt, and the like.

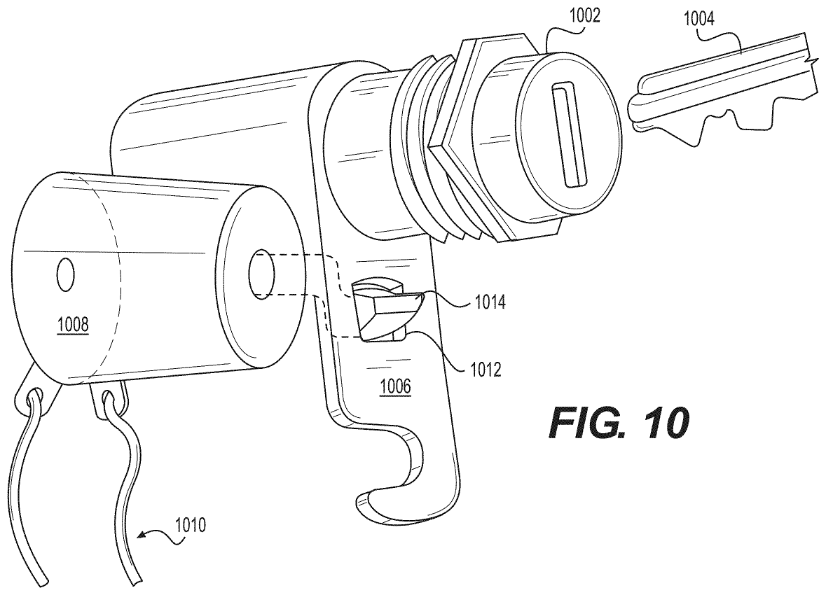

FIG. 10 illustrates a compound locking assembly according to embodiments of the disclosed technology. Referring to FIG. 10, the compound locking assembly includes a mechanical lock 1002, which may be operated by a physical key 1004 to rotate a latch 1006 into a corresponding notch, such as notch 204 in handle 302 of deck assembly 102, as shown in FIG. 3.

Referring again to FIG. 10, the compound locking assembly may also include an electric lock 1008, which may receive power through electrical cables 1010, and which may be operated using an electronic key, fob, remote control, or the like. When operated, the electric lock 1008 may insert a tab 1014 into an opening 1012 formed in the latch 1006 of the mechanical lock 1002, thereby preventing operation of the mechanical lock 1002.

In some embodiments, the electric lock 1008 may operate in parallel with the mechanical lock 1002. In such embodiments, the electric lock 1008 may insert the tab 1014 into a notch in the deck assembly. In such embodiments, both locks 1002, 1008 must be opened to release the deck assembly.

In some embodiments, the tab 1014 of the electrical lock 1008 may have multiple stops. In one of the stops, the tab 1014 engages the latch 1006 of the mechanical lock 1002, thereby preventing its operation, as illustrated in FIG. 10. In another of the stops, the tab 1014 engages a notch in the deck, thereby preventing its removal, as described above. In still another one of the stops, the tab 1014 engages neither the latch 1006 nor the deck assembly, thereby permitting operation of the mechanical lock 1002, and removal of the deck assembly.

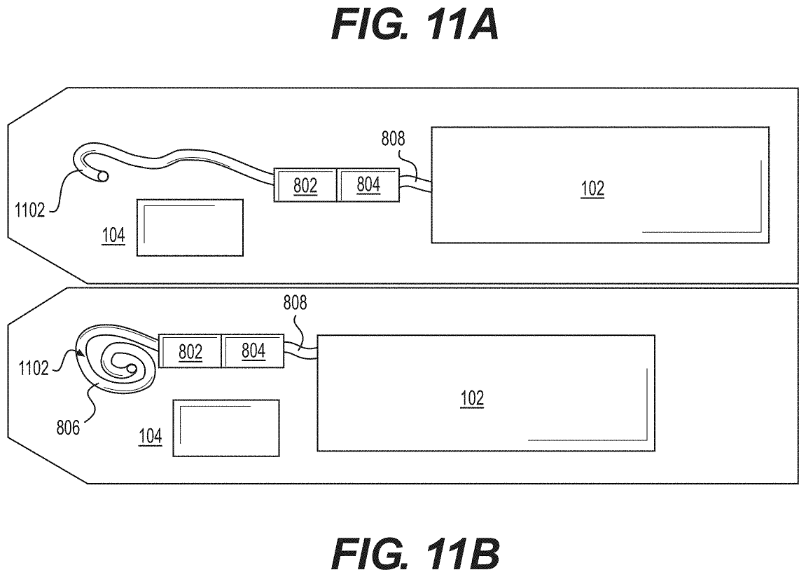

In embodiments that include an electrical power cable, the scooter may include a mechanism to retain and protect the cable when the deck assembly is installed. FIGS. 11A, B illustrate one such mechanism according to embodiments of the disclosed technology. In FIGS. 11A, B the mechanism is illustrated for the electrical cables 806, 808 and electrical connector 802, 804 of FIG. 8. However, the mechanism may be employed with any electrical cable and electrical connectors.

FIGS. 11A-B are top views of the scooter, with the rear of the scooter at the left. FIG. 11A illustrates a portion of the scooter 100 during removal or installation of the deck assembly 102. The battery pack in the deck assembly 102 is electrically coupled to the motor 108 by the electrical cables 806, 808 and the electrical connectors 802, 804. As shown in FIG. 11A, during installation or removal of the deck assembly 102, one or both of the electrical cables 806, 808 are extended to facilitate installation and removal, and to provide easy access to the electrical connectors 802, 804. A retention device 1102 permits this extension of the electrical cables 806, 808.

When the deck assembly 102 is installed in the frame 104 of the scooter 100, the retention device 1102 retracts, guides, organizes, and stores the loose portions of the electrical cables 806, 808, as shown in FIG. 11B. For example, the electrical cables 806, 808 may be retracted into a channel (not shown) formed in the frame 104 of the scooter 100. The retention device 1102 may be implemented as a spring-loaded device, for example such as a winding mechanism or the like. The winding mechanism may be similar to that used in spring-loaded tape measures, with the electrical cables 806, 808 taking the place of the tape. One benefit of this mechanism is that a technician working on the scooter does not have to manually feedback the slack in the electrical cables 806, 808, that results from the removal of the battery pack. When retracted, the electrical cables 806, 808, and the electrical connectors 802, 804, are protected from pinching, wear, and the like.

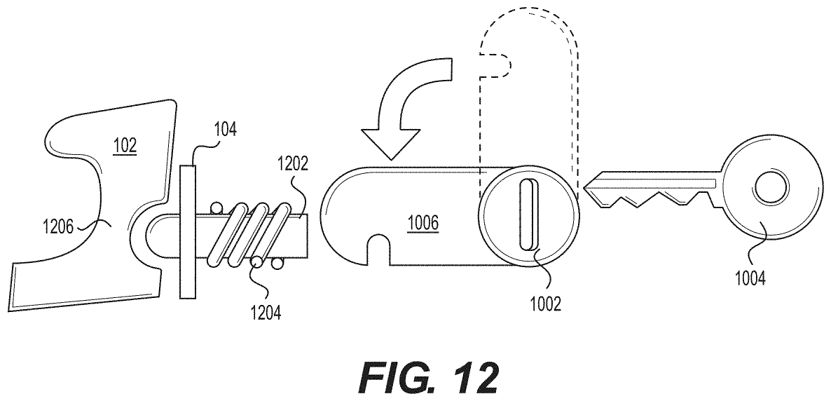

In some embodiments, the latch that retains the deck assembly 102 within the frame 104 of the scooter 100 may be hidden within a structure such as the frame 104 or the housing 110 of the scooter 100 so that it cannot be seen, and to protect the latch from damage. One such embodiment is illustrated in FIG. 12. The embodiment of FIG. 12 is illustrated for the mechanical lock 1002, physical key 1004, and latch 1006 of FIG. 10. However, the described embodiment may be employed with any lock, key, and latch, or with a keyless latch where the lock and key are replaced by a knob or the like.

Referring to FIG. 12, the described embodiment also includes a pin 1202 and a spring 1204 that biases the pin 1202 against the frame 104. When the lock 1002 and key 1004 are used to rotate the latch 1006 downward into a locked position, the latch 1006 forces the pin 1202 through a hole in the frame 104 into a notch 1206 formed in the deck assembly 102, thereby retaining the deck assembly 102 within the frame 104. When the lock 1002 and key 1004 are used to rotate the latch 1006 upward into an unlocked position, the spring 1204 backs the pin 1202 out of the notch 1206 so the deck assembly may be removed.

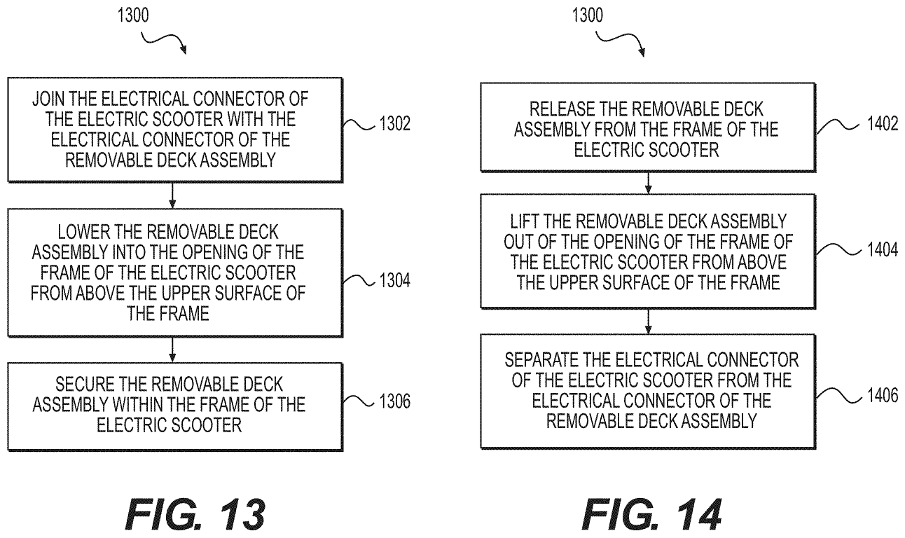

FIG. 13 illustrates a process 1300 for a user to install a removable deck assembly into an electric powered vehicle according to embodiments of the disclosed technology. While elements of the process 1300 are described in a particular sequence. It should be understood that certain elements of the process 1300 may be performed in other sequences, may be performed concurrently, may be omitted, or any combination thereof.

Referring to FIG. 13, the user may join the electrical connector of the electric powered vehicle with the electrical connector of the removable deck assembly, at 1302. The connectors may be joined as described above. The user may lower the removable deck assembly into the opening of the frame of the electric powered vehicle from above the upper surface of the frame, at 1304, for example as described above. The user may secure the removable deck assembly within the frame of the electric powered vehicle, at 1306, for example as described above.

FIG. 14 illustrates a process 1400 for a user to remove a removable deck assembly from an electric powered vehicle according to embodiments of the disclosed technology. While elements of the process 1400 are described in a particular sequence, it should be understood that certain elements of the process 1400 may be performed in other sequences, may be performed concurrently, may be omitted, or any combination thereof.

Referring to FIG. 14, the user may release the removable deck assembly from the frame of the electric powered vehicle, at 1402, for example as described above. The user may lift the removable deck assembly out of the opening of the frame of the electric powered vehicle from above the upper surface of the frame, at 1404, for example as described above. The user may separate the electrical connector of the electric powered vehicle from the electrical connector of the removable deck assembly, at 1406 for example as described above. Spatially relative terms such as “under,” “below,” “lower,” “over,” “upper,” and the like, are used for ease of description to explain the positioning of one element relative to a second element. These terms are intended to encompass different orientations of the device in addition to different orientations than those depicted in the Figures. Further, terms such as “first,” “second,” and the like, are also used to describe various elements, regions, sections, etc. and are also not intended to be limiting Like terms refer to like elements throughout the description.

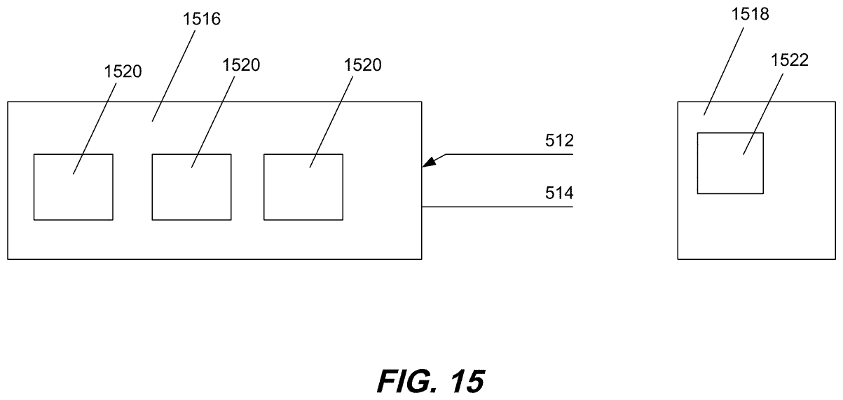

In one embodiment, illustrated in FIG. 15, electric vehicles 1516 are provided with systems and methods for vehicle security without a hardware secure element 1510. Hardware secure elements 1510 usually allow for the storage of private keys 1512, which are used to sign and encrypt data. In one embodiment the present invention removes the dependency on a hardware secure element 1510 as part of the whole security system.

Private keys and private key pairs (collectively 1512 and 1514) are used to cryptographically secure sensitive information. private keys 1512 can be used to decrypt, encrypt, or sign data. the corresponding public key 1514 can be used to decrypt or verify the signature of the data signed by its private key. public keys cannot be used to encrypt or sign data.

As a non-limited example, as used herein a vehicle 1516 is a means of carrying or transporting something including but not limited to an EV motor vehicle 1516, including but not limited to a scooter, skateboard, skates, and the like.

As used herein an encryption key is a piece of information that determines the functional output of a cryptographic algorithm. For encryption algorithms, a key specifies the transformation of plaintext into ciphertext, and vice versa for decryption algorithms. Keys also specify transformations in other cryptographic algorithms, such as digital signature schemes and message authentication codes.

As used herein, the cloud 1518 is a global network of servers, each with a unique function. The is not a physical entity, but instead is a vast network of remote servers around the globe which are hooked together and meant to operate as a single ecosystem. These servers are designed to either store and manage data, run applications, or deliver content or a service such as streaming videos, web mail, office productivity software, or social media. Instead of accessing files and data from a local or personal computer, you are accessing them online from any internet-capable device—the information will be available anywhere you go and anytime you need it. In the case of this embodiment the cloud 1518 is securely storing and generating public key and private key pairs for each component in the vehicle 1516.

As non-limiting examples, there are four different methods to deploy 8 resources.

These include: a public cloud 1518 that shares resources and offers services to the public over the Internet; a private cloud that isn't shared and offers services over a private internal network typically hosted on-premises; a hybrid cloud that shares services between public and private clouds depending on their purpose; and a community cloud 1518 that shares resources only between organizations, such as with government institutions.

In one embodiment, system 10 is coupled to the cloud 1518.

As used herein, a local area network (LAN) is a network that interconnects within a limited area such as a residence, school, laboratory, university campus or office building. By contrast, a wide area network (WAN) not only covers a larger geographic distance, but also generally involves leased telecommunication circuits. Ethernet and Wi-Fi are two common technologies in use for local area networks. Historical network technologies include ARCNET, Token ring, and AppleTalk.

As a non-limiting example, a wide area network (WAN) is a network that exists over a large-scale geographical area. A WAN connects different smaller networks, including local area networks (LANs) and metro area networks (MANs). This ensures that computers and users in one location can communicate with computers and users in other locations. WAN implementation can be done either with the help of the public transmission system or a private network.

As a non-limiting example, system 10 is coupled to the cloud. This can be achieved via GSM, WiFi, satellite, a mobile device and the like.

Other wireless standards that are specifically designed for IoT devices are becoming available such as Lora, NB-IOT and LTE-M, and the like.

As a non-limiting example, in one embodiment one or more hardware elements 1510 of the vehicle 1516 has public keys 1514 stored therein. Secure encryption is not put on the hardware elements 1510.

A vehicle 1516 consists of one or more in individual components 1520. Individual components 1520 of the vehicle 1516 are given an Acton Unique Identifier (AUIDs). When a vehicle 1516 is activated the first time, a unique public key 1514 and private key 1512 pair are generated by the cloud. AUIDs, public key and private keys 1514 and 1512 are then stored in the cloud. Each component stores its AUID and public key in persistent memory within the component thus eliminating theft of private keys 1512.

For selected components 1520 of the vehicle 1516, the cloud 1518 produces a unique private key 1512 and a public key 1514. As a non-limiting example, with the present invention, private keys 15112 are secure and in the cloud. They cannot be taken from the vehicle 1516. Non-limiting examples of vehicle 1516 components 1520 with public keys 1514 include but are not limited to: IOTA, the battery, motor controller, and the like.

As non-limiting examples, a simple electric vehicle 1516 can include a battery; vehicle control unit (motor controller), and IoT gateway. Each of these components 1520 is given an AUID. Additional components 1520 include but are not limited to vehicle locks; dashboards; helmets; docking stations; and the like.

As non-limiting examples, selected vehicle components 1520 have unique IDs with a unique identifier. These components 1520 are given a unique key pair. As a non-limiting example, the private key 1512 is securely stored in the cloud. An associated public key 1512 is stored in the vehicle components 1520. Communication in the cloud 1518 can be authenticated with the vehicle 1516 through the components 1520 that have public keys.

As a non-limiting example of authentication steps, public keys 1514 are passed to the vehicle 1516, e.g., vehicle components 1520. The private key 1512 is stored in the cloud, and the public key 1514 is transferred to a respective vehicle component.

As a non-limiting example, when the vehicle 1516 connects to the server 1522, it tells the server 1522 it has components 1520 A, B, and C. The System looks up in an associated database and generates an activation message composed of multiple parts, each part signed with the private key 1512 that corresponds to the AUID of the vehicle component A, B, or C 1510. When the activation message is received by the vehicle 1516, the individual components 1520 A, B, and C will decrypt and verify their parts of the message. If anyone component's message part fails verification, the vehicle 1516 will not activate.

As a non-limiting example, a secret key is not needed that unlocks the entire scoter. Instead, the system creates components 1520 are identified as being unique with associated keys.

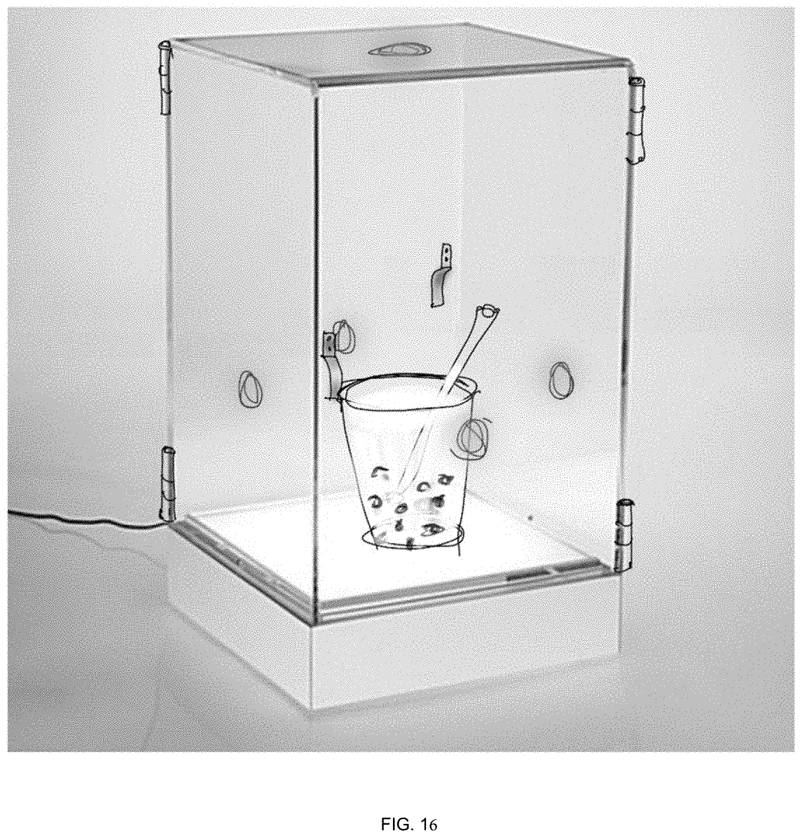

As illustrated in FIG. 16, in one embodiment fleets of vehicles are used to distribute information between vehicles in the fleet. As a non-limiting example, individual fleet vehicles have two wireless communication networks. The first is any kind of cloud 1518 connectively. The second one is any kind of local wireless communication.

When vehicles communicate with the cloud, they report their status occasionally. When they report status, they report the presence of other fleet-vehicles that they have detected on local wireless. As a non-limiting example, this status message can then be communicated with other fleet vehicles IDs that are within local communication. This provides information about the location of fleet vehicles, which can be used to reduce theft and increase fleet availability.

As a non-limiting example, data can be distributed to the fleet by seeding it to only certain vehicles, and these vehicles that receive the communications then communicate with other vehicles. Data that could be sent includes, but is not limited to updates, navigation information, vehicle configuration, secure one-time-keys. This mechanism decreases fleet-wide data-usage and improves fleet operation.

As a non-limiting example, a vehicle 1516 can detect, via local wireless communication, other vehicles, report their presence to the cloud, and the can then determine if another vehicle 1516 is located within a selected proximity. The cloud 1518 can then determine if the reporting vehicle 1516 can communicate data to the other vehicle. The cloud 1518 can then send a one-time use session key to the vehicles, allowing them to communicate securely.

When a vehicle 1516 communicates with the cloud 1518 that it sees another vehicle, it sends this message up to the cloud. The cloud 1518 can use this vehicle 1516 presence information to disable vehicles, track stolen vehicles, locate missing vehicles, and the like.

Fleet vehicles are vehicles operated by an entity that provides them for public or private use to individuals or employees. A fleet is a group of one or more Fleet Vehicles that an operator makes available for use. Private vehicles are vehicles operated by individuals for their own use.

In one embodiment, this invention can be used with both fleet and individual vehicles. If individual or fleet Operators of EV include their vehicle 1516 in this system, the benefits of lost vehicle 1516 discovery, reduced data usage, and the like can be extended across fleets and individuals. In this way, the fleet vehicles of Operator A can look for a stolen fleet vehicle 1516 of Operator B, while a private vehicle 1516 operated by individual C can receive software update data from Operator A's fleet.

When misplaced or stolen fleet or individual vehicles are located, the owner and/or authorities can be notified.

The coronavirus has been divided the virus into a plurality of sub-groupings, including but not limited to: 229E (alpha); NL63 (alpha); 0C43 (beta); HKU1 (beta); MERS-CoV; SARS-CoV; SARS-CoV-2; and the like.

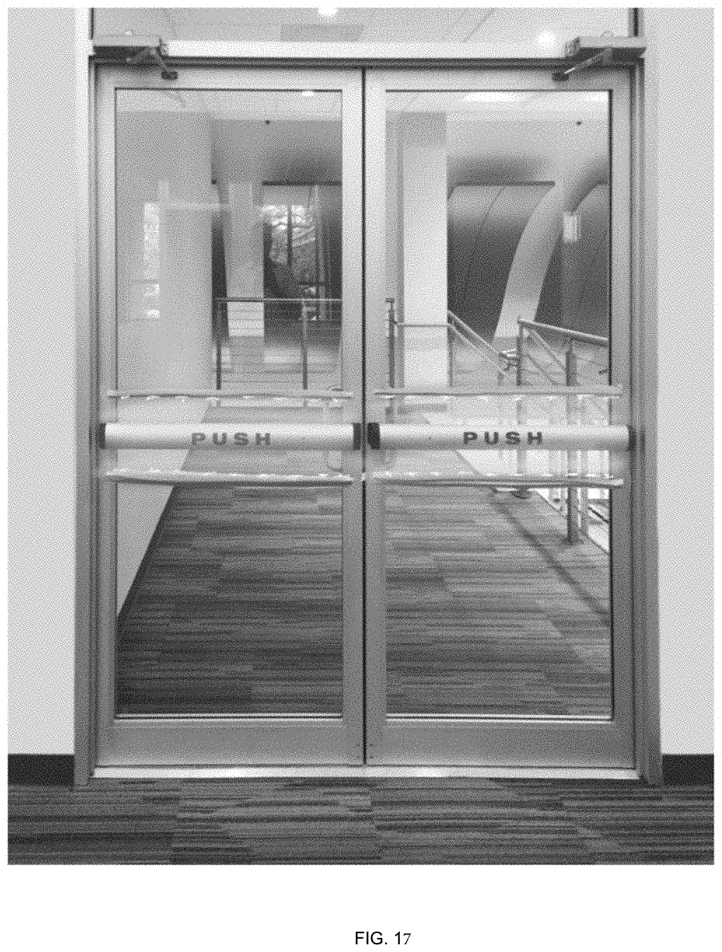

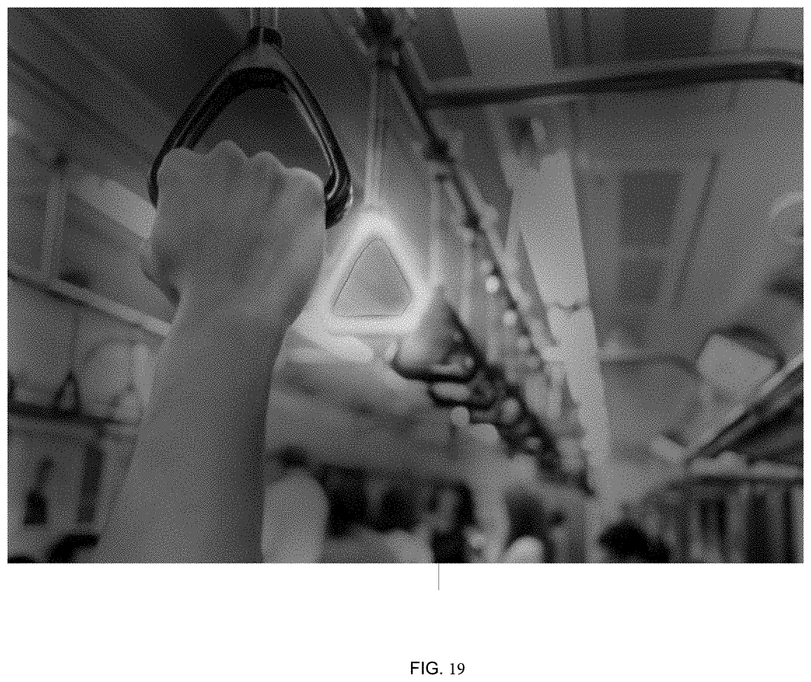

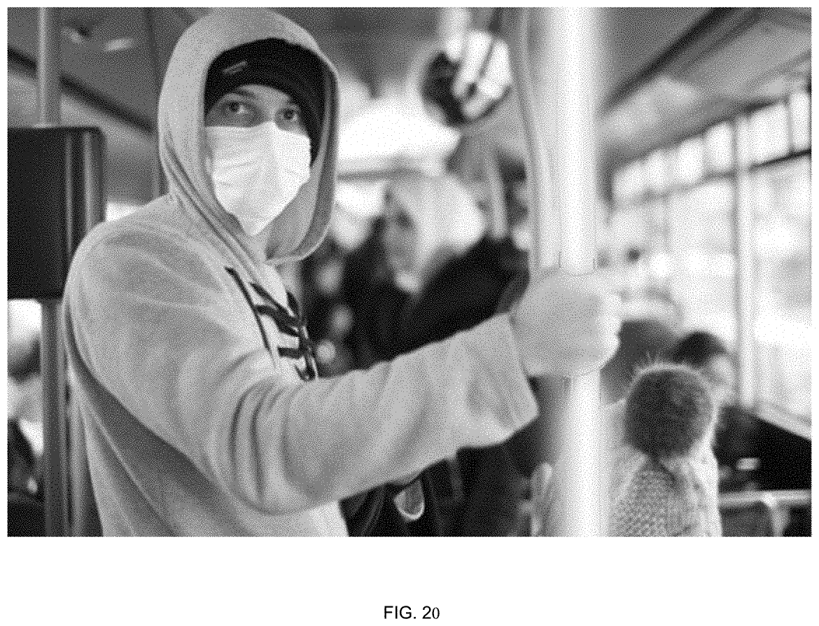

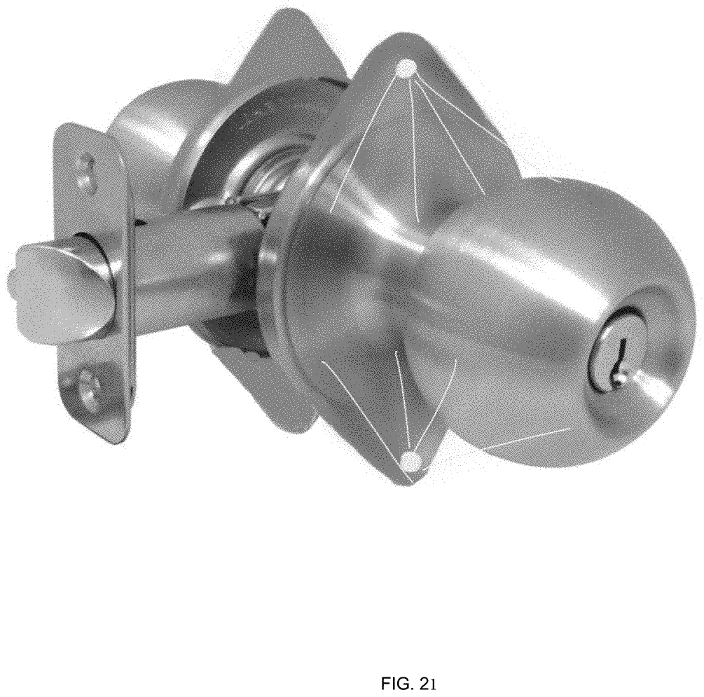

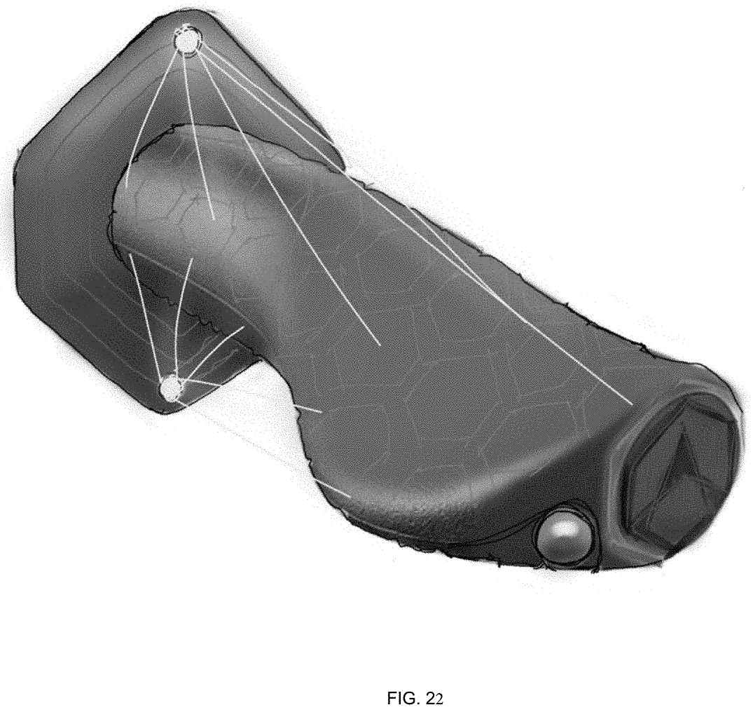

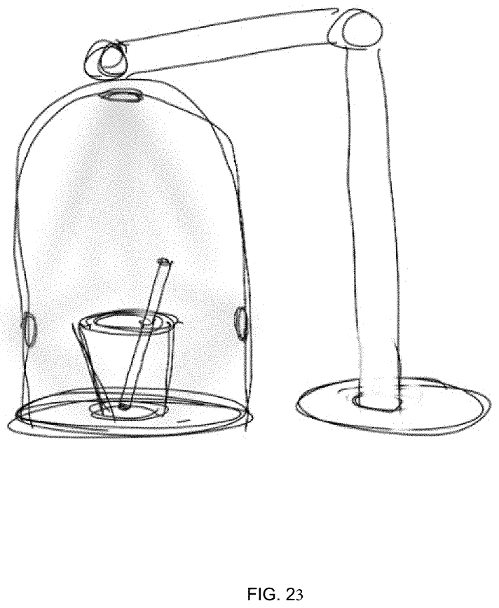

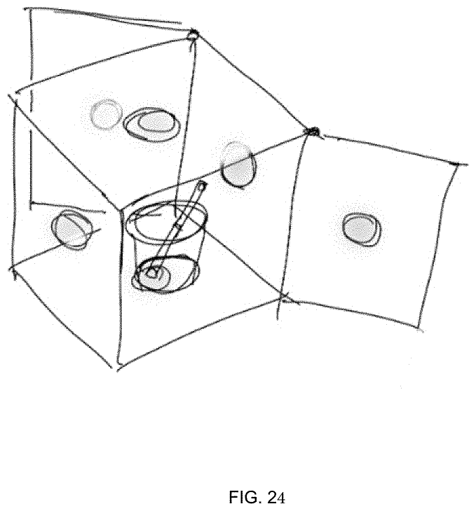

Referring to FIGS. 17-24 As non-limiting examples, UVC lights 1610, 200-400 nm, can be provided, as well as any light that kills pathogens, including but not limited to light with intensity for a certain amount of time, lights 1610 that shine on the handlebar, or a plastic handle bar that is made with lights 1610 at an interior. As a non-limiting example, a plurality of UVC lights 1610 can be positioned at a distal end of a handlebar, see FIG. 24, to provide that UVC light is incident on a total surface where there can be human contact. In one embodiment, three UVC lights are included. In one embodiment, the handlebar includes a distal end with a plurality of UVC light sources positioned there to produce light incident on a hand grip portion. The UVC light sources can be coupled to a controller and a power source. As a non-limiting example, three or more UVC lights 1610 can be used, positioned and configured to provide illumination of substantially all of a surface that can be in contact with a human electric vehicle operator.

UVC lights 1610 can be used for different surface to kill the coronavirus, in one embodiment any surface of a vehicle, included but not limited to electric powered vehicle 100s, electric vehicle, non-electric vehicle, and the like. that an operator or rider is in contact with, including but not limited to handlebars 116, as well as any surfaces that more than one-person use, including but not limited to: public transport surfaces, airline surfaces, bus handles, car handles, knobs, door knobs include UVC lights 1610, disinfectant 1612 or the like, is provided that kills a virus and other pathogens.

UVC source 1610 can be used for different surface to kill the coronavirus, in one embodiment any surface of a vehicle, included but not limited to electric powered vehicle 100, electric vehicle, non-electric vehicle, and the like. that an operator or rider is in contact with, including but not limited to handlebar 116, as well as any surfaces that more than one-person use, including but not limited to: public transport surfaces, airline surfaces, bus handles, car handles, knobs, door knobs include a UVC light 1610, disinfectant or the like, is provided that kills a pathogen, including but not limited to the coronavirus.

In one embodiment any type of handle device that people touch, externally with UV, or internally with the light shine inside.

As non-limiting examples, door knobs include but are not limited to: entrance door handles typically used on exterior doors, and include keyed cylinders; privacy door handles typically used on bedrooms and bathrooms; while they are lockable; passage knows such as hall or closet, these do not lock and are used in hall or closet doors.

As non-limiting examples, any type of handle device that people touch, externally with UVC source 1610, or internally with the light shine inside.

In one embodiment, UVC source 1610 can be used with buttons touched by people. These buttons are including difficult to avoid, which is part of the reason why push buttons can be crawling with germs. Further, ubiquitous buttons, are found on ATMs, elevators, telephones and drink machines, among other things, are located in areas that are not often cleaned and disinfected to kill bacteria and viruses.

In another embodiment, any surface of a vehicle that an operator or rider is in contact with can be made from an antibacterial/antimicrobial material (hereafter “sanitizing layer/coating”), or an associated exterior surface, where the operator or rider contacts, including but not limited to grips, brake levers, and the like, can be treated with a sanitizing layer/coating. As a non-limiting example, an actual paint, applied at the factory, can include these elements. In one embodiment, the entire vehicle can be covered with the sanitizing layer/coating.

In one embodiment, ‘stickers’, which are essentially thin pieces of plastic with adhesive backing, can be attached to selected areas of the vehicle, including but not limited to brake levers, bells, throttles, other parts of high contact and the like.

In one embodiment any type of handle device that people touch, externally with UV, or internally with the light shine inside. As non-limiting examples, door knobs include but are not limited to: entrance door handles typically used on exterior doors, and include keyed cylinders; privacy door handles typically used on bedrooms and bathrooms; while they are lockable; passage knows such as hall or closet, these do not lock and are used in hall or closet doors.

As non-limiting examples, any type of handle device that people touch, externally with UVC light source 1610, or internally with the light shine inside. In one embodiment, UVC light source 1610 can be used with buttons touched by people. These buttons are difficult to avoid, which is part of the reason why push buttons can be crawling with germs. Further, ubiquitous buttons, are found on ATMs, elevators, telephones and drink machines, among other things, are located in areas that are not often cleaned and disinfected to kill bacteria and viruses.

In another embodiment, any surface of a vehicle that an operator or rider is in contact with can be made from the sanitizing layer/coating. In one embodiment, energy is supplied to the surface to equal to or exceed 60 degrees F.

In one embodiment, irradiation can form the irradiation control part of the UVC lights 1610 of a luminous intensity distribution figure regional area and the irradiation state of control for distance light. The irradiation control and irradiating state is provided so a distance of the UVC light with the regional area of luminous intensity distribution figure is controlled. This can be achieved by being at least divided into a plurality of subregions in electric vehicle 10 handlebar 116 width direction, and form. In one embodiment there is an adjustment of the luminous intensity from the UVC lights 1610 corresponding with each of the several part regions. In one embodiment, three or more UVC lights 1610 are used, in order to transmit illumination in surface area of up to 360 degrees.

As a non-limiting example, an amount or dosage of UVC light that is required to disinfect most pathogens is one or more of: 15 mJ/cm2 to 20 mJ/cm2; 16 mJ/cm2 through 19 mJ/cm21; 18 mJ/cm2; and the like. A positioning of the UVC light sources 1610 and/or shaping of UVC light reflectors to reflect the incident UVC light source 1610 is provided to be incident and cover a surface of the structure that needs to be disinfected, ideally for a handlebar 116 grip, at least 95%, at least 75%. The positioning and/or shaping is also selected to reduce the running time of each UVC source 1610. As a non-limiting example, dosage calculations are determined by one or more of: UVC light source 1610 strength; distance from the UVC light source 1610 to the surface to be disinfected; effective beam width (angle of light emission), each UVC light source 1610 having a protective lens. The lens determines the angle of emission, which can be 120-160 degrees for a total of 360 degrees of illumination. As non-limiting examples, the duration of illumination can be 2 minutes, 90 seconds, 75 second, as well as a range of 45 to 90 or 120 second is provided.

In one embodiment one or more are used: multiple UVC light sources 1610, at a same distance, can cover a larger area for the same on time duration. In one embodiment, a same area is covered by the lights 1610 and there is a decrease in the time duration that the UVC light sources 1610 are one.

As a non-limiting example, a calculation of UVC dosage is as follows: UVC Emission Strength (mW)×0.001=UVC Emission Energy (mJ/s) 40 milliwatts; and UVC Dosage (mJ/cm2)=UVC Emissions Energy (mJ/s)/Surface Area (cm2)*UVC On time (s)=can be 15-20 mJ/cm2; 18-20 mJ/cm2; 18-19 mJ/cm2; and 19 mJ/cm2

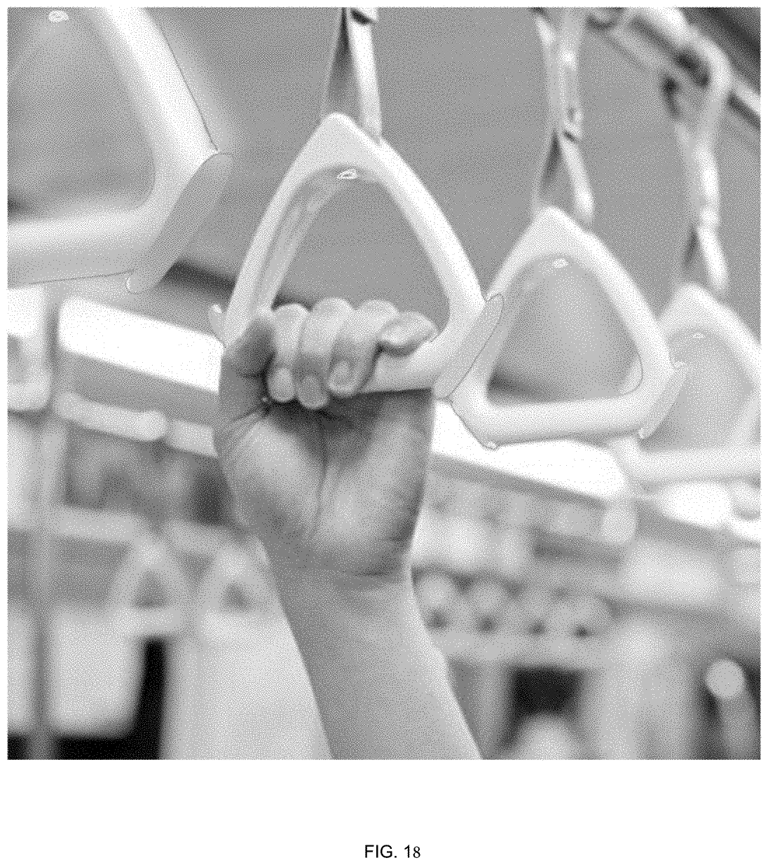

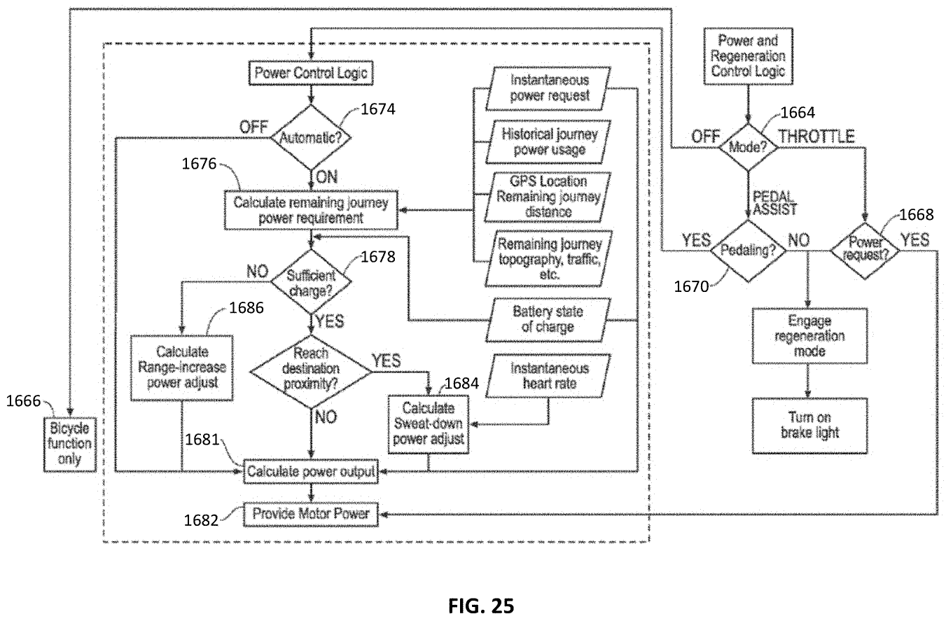

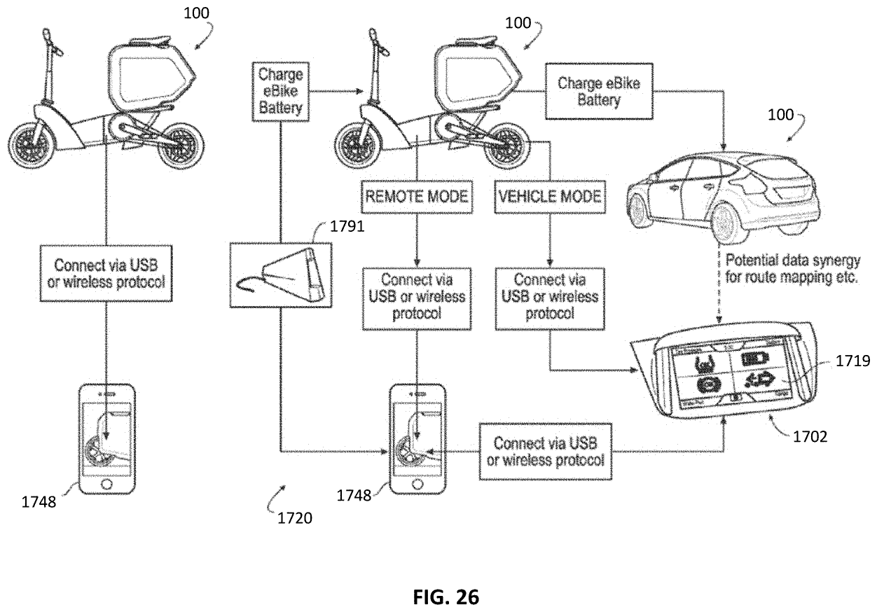

As illustrated in FIGS. 25 and 26, the electric powered vehicle 100 may include a controller in communication with the motor 1642 and the brake light 1643. The controller may be configured to illuminate the brake light 1643 when the gear 1650 drives the belt 1654, i.e., when the electric powered vehicle 1600 coasts. For example, with reference to FIG. 18, the rear brake light 1643 may be in communication with the computing device 1628 of the electric powered vehicle 100. The computing device 1628 may be programmed to illuminate the brake light 1643 when the gear 1650 drives the belt 1654. In other words, for example, in the event the driver stops pedaling and the electric powered vehicle 100 coasts, the gear 1650 drives the belt 1654 and motor 1642 generates energy for charging the battery 1626, in such events, the computing device 1628 may detect that the gear 1650 is driving the belt 1654 and, in response, provides an instruction to illuminate the brake light 1643.

With reference to FIG. 26, the computing device 28 may be programmed with a power regeneration control logic. As shown at block 1664, the power regeneration control logic may have three modes. Specifically, the power regeneration control logic may be turned off, may be operated in a throttle mode, or may be operated in a pedal-assist mode. The pedal-assist mode may be referred to as pedal or power-assist mode. The purpose of the pedal assist mode, for example, may be to comply with rules such as European Union directive 2002/24/EC and/or EN15194 for road-legal use of electric powered vehicle 100.

When the power regeneration control logic is turned off, the electric powered vehicle 100 may be propelled by manual input with the crank 1656 and is not powered with the motor 1642, as shown in block 1666. In the throttle mode, the electric vehicle 100 may be propelled by the motor 1642 and controlled independently of the manual input from the crank 1656. As shown at block 1668, when power is requested, i.e., with the input operated by the driver, the power regeneration control logic provides power to the motor 1642. For example, the input may be variably operated to vary the power to the motor 1642. As shown at block 1668, if power is not requested, the power regeneration control logic operates in a regeneration mode. In the regeneration mode, the rear wheel 1622 of the electric powered vehicle 100 slows and a brake light is activated.

When the power regeneration control logic is turned to pedal-assist mode, the electric powered vehicle 100 may be propelled by both the motor 1642 and by manual input from the crank 1656. As shown at block 1670, only if the driver pedals the crank 1656, can the electric powered vehicle 100 be propelled with the assistance of the motor 1642 to complement the power delivered manually by the driver. The exact amount of power assistance is calculated by the computing device 1628. When in the pedal-assist mode, when the crank 1656 is not pedaled, the power regeneration control logic operates in a regeneration mode. In the regeneration mode, the rear wheel 1622 of the electric powered vehicle 100 slows and the brake light is activated.

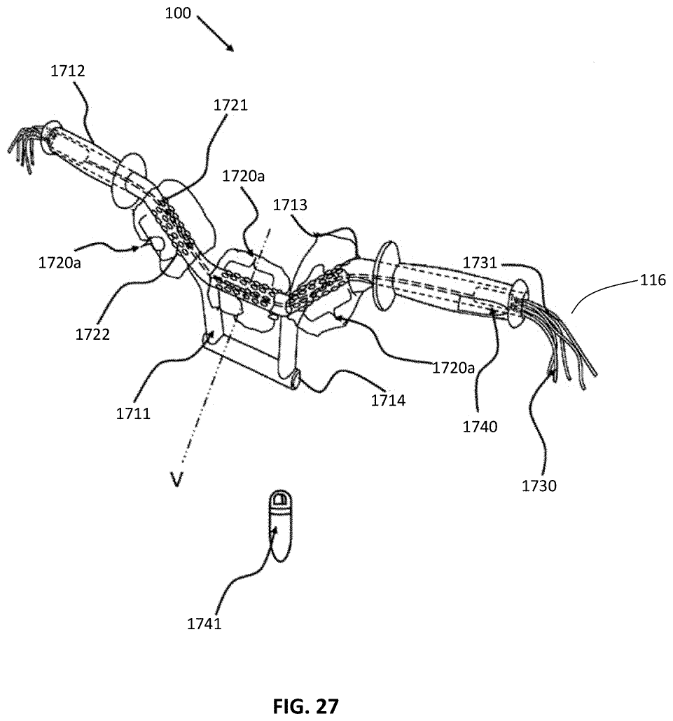

FIG. 27 is a partial view of an embodiment of the high visibility safety lighting system, wherein a UVC light source 1610 illumination segments are integrated within a handlebar 116 assembly of an electric powered vehicle 100. The high visibility safety lighting system is shown integrated within a handlebar 116 assembly 1711 of an external frame of a vehicle. Handlebar assembly 1711 includes hand grip portions 1712, a u-bar attachment 1713, and a base attachment 14 appointed for attaching handlebar 116 assembly 1711 to electric powered vehicle 100. At least one illumination segment 1720 (1720a-1720n) is incorporated within handlebar 116 assembly 1711.

As a non-limiting example, there is a plurality of illumination segments 1720a-1720n provided; herein three such segments 1720a located in u-bar attachment 1713. At least one UVC light 1610 is located within said illumination segment 1720a-1720n powered by a power source (battery) controlled by a power control for supplying current to the UVC light 1610 and activating same to emit light from illumination segments 1720a-1720n.

Illuminating segments 1720a herein are provided as a UVC light source 1610 illuminating segments, wherein a plurality of apertures 21 are located within each of the segments 1720a having a UVC light source 1619 22 integrated therein. UVC light sources 1610 may be sequenced intermittently to generate moving, blinking, flashing or cascading light. The high visibility safety lighting system may be integrated into the frame of a plethora of vehicles, including a motorcycle, scooter, or other motor driven cycle-like device, stroller, walker, or child's toy, ski poles, etc., or an electric powered vehicle 100 for an adult or child. At least one of illumination segment 1720a-1720n can be integrated within handlebar 116 assembly 1711 of the frame. In addition, the body assembly of the frame preferably includes at least one illumination segment 1720n integrated therein.

The high visibility safety lighting system preferably includes one or more on-board batteries as the power supply means. The power control attached to the power supply means may be activated a number of different ways, including: (i) a card reader 40 and a key 41 having a card tag that activates the card reader and provides instant activation of the current to the lighting source in illumination segments 1720 without concern for water effecting the power control; (ii) a pressure sensor located within an interior/seat of the vehicle for supplying current to the UVC light 1610; (iii) a light sensor engaged with the power control attached to the power supply means for automatically activating/deactivating the current supply to the UVC light 1610; (iv) an “on”/“off” power switch; or (v) one or more acceleration detecting means, a delay generating circuit means, and a microprocessor.

In one embodiment, illustrated in FIG. 27 is a cross-sectional view of the handlebar 116 assembly taken along line V of FIG. 27, showing a cross-section of a UVC illumination segment 1720a. Rumination segment 1720a is preferably cylindrical as shown, but the shape and size is determined based upon the location on the frame of the vehicle. A plurality of apertures 1721 are integrated into illumination segment 1720a and UVC sources 1619 therein. In one embodiment, UVC sources 1619 and visa vie apertures 21 traverse substantially the entire circumference of the frame section, as shown, to yield 360 degrees of light emission, especially when utilized on the handlebar 116 assembly. Alternatively, UVC sources 1610 and apertures 17 21 cover the outer show surface of the segment 1720a. UVC illumination segment 1720a may include at least one acceleration sensor shown at 1723. A battery 1724, which is preferably rechargeable, powers the device when the optional on/off switch 1725 is turned on. The battery 1724 powers the acceleration sensor 1723, the microprocessor 1726, the delay time generating circuit element 1727, which is optionally user adjustable, and a plurality of UVC sources 1610. The microprocessor 1726 receives an acceleration signal from the acceleration sensor 1723 when the electric vehicle 100 is moved. The microprocessor turns on the UVC sources 1610 land keeps them turned on for a time period set by the delay time generating circuit element 1727. The optional on/off switch at 1725 is used to turn off power.

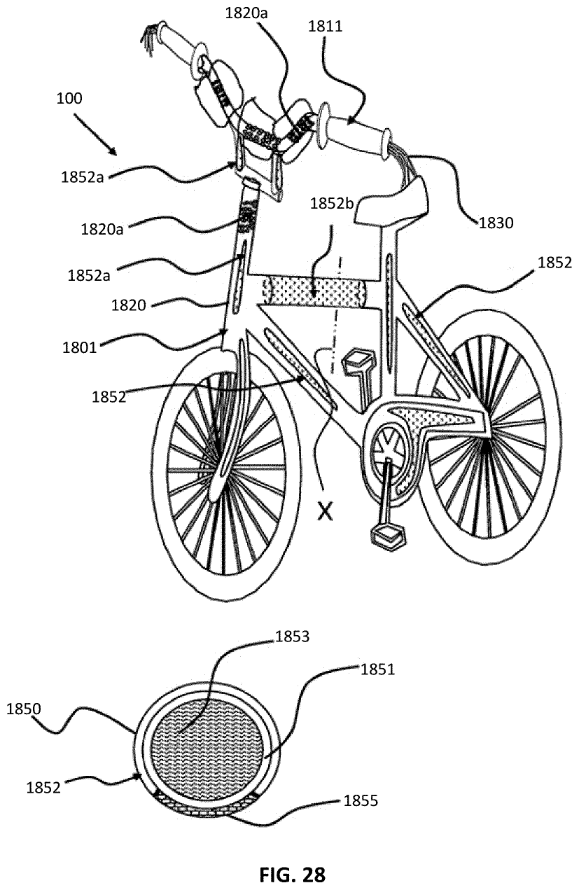

In one embodiment, shown in the schematic view of FIG. 28, electric powered vehicle 100 has a plurality of UVC light source 1610 illumination segments and transparent illumination segments integrated within the frame of the electric powered vehicle 100, shown generally at 100. Electric vehicle 100 includes a frame having a handlebar 116 assembly 17111 and body assembly 102. Herein handlebar 116 assembly 111 includes illumination segments comprising UVC illumination segments 11720a, along with illuminated streamers. UVC lights 1610 illumination segments 11720b are also preferably in body assembly 102 of bike 100. In addition, at least one illumination segment is transparent/or includes a transparent window 150 arranged with an opaque section 1755 and comprises an inlay cavity 1851 therein to form a transparent illumination segment 1852. A plurality (shown herein as two) transparent illumination segments 1852a are provided within the handlebar 1816 assembly 1811; while a plurality of transparent illumination segments 1852b is shown in the body assembly of the bike 1800 (shown as shaded regions). The UVC light 1610 utilized in transparent illumination segment 1852 comprises an electroluminescent wire or strip 1853 that is housed within inlay cavity 1851 of transparent illumination segment 1852a, 1852b. A plurality of transparent illumination segments 1852b is provided within body assembly 1802 of the electric powered vehicle 1800's frame, indicated by shaded regions. Transparent illumination segments 1852a, 1852b-152 n may be located anywhere on body assembly 1802 or/and on handlebar 116 assembly 111.

In an alternative embodiment, virtually the entire electric powered vehicle 100 may be composed of transparent material; that is to say, the body assembly 1802 and handlebar 116 assembly 111 of the frame are substantially composed of a transparent material constructed with an inlay channel therein appointed for housing the UVC light 1610 comprising an electroluminescent light strip. The inlay channel and electroluminescent light strip extend within the electric powered vehicle 100's frame so that a substantial portion of the frame emits light, thereby forming a lighted outline of the frame in dimly lit surroundings.

Transparent display/window can be a plethora of shapes and sizes, and may include large regions within illumination segment 1820. Transparent displays can be a plethora of shapes, sizes, designs, characters, indicia, and so on. Transparent displays may be specific characters and advertise various organizations. Moreover, opaque sections and transparent displays may be of any size, and may merely be speckles located along the entire surface of illuminated segments 1820, which in turn can be on the entire surface of frame 1801.

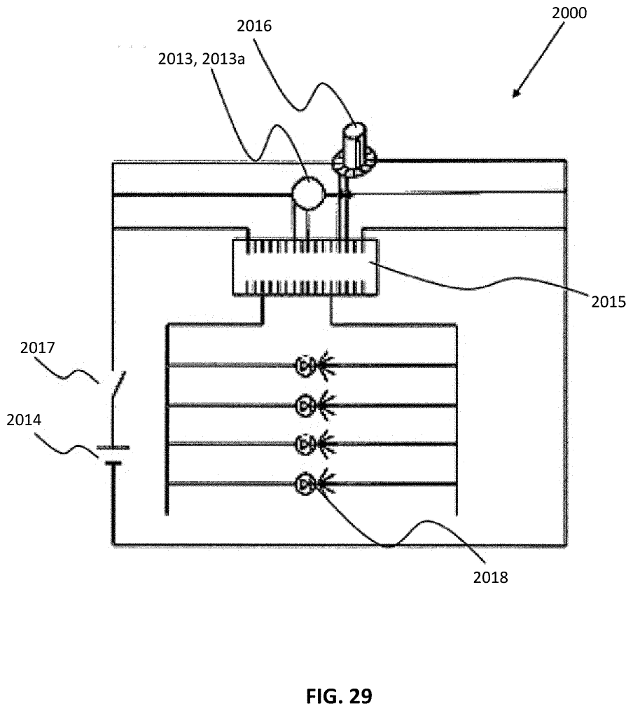

FIG. 29 illustrates generally at 2000 a circuit of the high visibility safety lighting device wherein the acceleration sensor is utilized. Battery 2014 powers the high visibility safety lighting device through an on/off switch 2017. When switch 2017 is in the on position, power is supplied to acceleration sensor 2013 and optionally, a second acceleration sensor 2013a, delay time generating circuit 2016 and microprocessor 2015. When microprocessor 2015 receives an acceleration signal from the acceleration sensor 2013, the microprocessor applies battery supplied power to the UVC light 1610, including the plurality of UVC sources 1610 418. The UVC sources 1610 stay lit by the microprocessor for a pre-selected time period, as set by a signal from delay time generating circuit element 2016 to the microprocessor 2015.

Vehicles such as motorcycles, electric powered vehicle 100s, scooters, wheel chairs, and toy cycles used by children also have varying levels of acceleration and deceleration during their operating cycle. The accelerometer sensor picks up these accelerations and turns on the UVC sources 1610 for each of the acceleration detected. This, in combination with delay time generating circuit essentially illuminates the UVC sources 1610 during the entire operating period of the vehicle. Even when the motorcycle, electric powered vehicle 100, scooter, wheel chair, or a toy cycle is stopped at a stop light, traffic crossing, or other locations, the UVC sources 1610 stay “on” for a pre-selected time period, regulated by the delay time generating circuit, thereby providing an increased margin of safety.

Optionally, the delay time generating circuit has a variable resistance or a timing control element that enables the user to set the delay time period. This delay circuit adjustment enables the user to select the time period for which the UVC sources 1610 will stay on and is particularly suitable when long time interval stoplights are encountered. Since the UVC sources 1610 are turned off at the end of the delay circuit time period, the battery life is conserved. Optionally, an on-off switch is provided adjacent to the battery connection to turn off the high visibility safety lighting device. This switch is used when the high visibility safety lighting device is removed from the vehicle or stroller and stored for a prolonged time period. Turning off the on-off switch disconnects all the electrical components of the high visibility safety lighting device.

Both analog and digital accelerometers suited for the high visibility safety lighting device are available in a variety of measurement configurations. Analog accelerometers provide an analog output, typically a current in the range of 4 to 1720 milliamps or an output voltage of −5V to +5V according to the g-force detected. Digital accelerometers output a pulsed high frequency waveform with a varying square wave pulse width and therefore, the frequency. A capacitive acceleration sensor uses a metal beam or micro-machined feature to produce a capacitance which changes with the acceleration of the device. A piezoelectric acceleration sensor uses a piezoelectric crystal mounted on a mass, and the piezoelectric voltage output is converted to acceleration. A piezo-resistive acceleration sensor has a beam or micro-machined feature whose resistance changes with acceleration. A Hall effect acceleration sensor converts motion to an electrical signal by the sensing of a changing magnetic field. A magneto resistive acceleration sensor detects changes in material resistivity in the presence of a magnetic field. More recently heat transfer acceleration sensors have been produced which track location of a heated mass during acceleration by sensing temperature. Several of these acceleration sensors react at high frequencies and therefore any mechanical vibration of the sensor is reported as a very high value of g-force. Mechanical spring and ball type accelerometers are also available. The reliability of such accelerometers is poor as compared to other accelerometers due to their bounce characteristics and ball sticking behavior. Accordingly, mechanical spring and ball type accelerometers are not preferred for applications of the safety lighting device. Digital accelerometers are highly reliable and are not damaged when the sensor is subjected to high g-forces. In addition, these sensors detect acceleration in more than one axis.

Analog devices Inc. produces digital output multi-axis accelerometers. These digital devices directly couple to a microprocessor to determine the acceleration or deceleration. For example, MEMS sensor ADXL202/JQC/AQC measures ±2 g, while sensor ADXL210/JQC/AQC measures ±10 g.

Silicon Devices Inc. produces digital output multi-axis accelerometers based on micro electro mechanical (MEMs) technology. A LIS3LVO2DQ sensor is a 3-Axis-±2 g/±6 g digital output low voltage linear accelerometer.

In one embodiment, a low profile amplified piezoelectric accelerometer is used. These have constant current low output impedance output combined with the ability to drive high load capacitance allows long runs of low-cost cable without degradation of data. As a non-limiting example, an accelerometer can have high natural frequency, a wide frequency range, a flat sensitivity vs. temperature response over the temperature range. and a low base strain sensitivity. As a non-limiting example an accelerometer is used that is magneto resistive a high frequency acceleration detector with a dynamic range of ±80 g.

As a non-limiting example, an accelerometer that is used is a micro machined silicon accelerometer with two silicon beams vibrating at different frequencies. In one embodiment, their frequency difference is used to determine a g-value. Such an accelerometer is capable of detecting ±80 g and produces a digital wave output, whose frequency depends on the g-value.

It is to be understood that the present disclosure is not to be limited to the specific examples illustrated and that modifications and other examples are intended to be included within the scope of the appended claims. Moreover, although the foregoing description and the associated drawings describe examples of the present disclosure in the context of certain illustrative combinations of elements and/or functions, it should be appreciated that different combinations of elements and/or functions may be provided by alternative implementations without departing from the scope of the appended claims. Accordingly, parenthetical reference numerals in the appended claims are presented for illustrative purposes only and are not intended to limit the scope of the claimed subject matter to the specific examples provided in the present disclosure.

What is claimed is:

1. An electric vehicle, comprising:
a steering element that can include a handle bar element, the steering element including, hand grip portion, and including at a distal end a plurality of UVC light sources producing light that is incident on the hand grip portion, the UVC light sources coupled to a controller and a power source;
a platform configured to support a rider of the electric vehicle;
at least one front wheel and at least one rear wheel;
an electric motor configured to provide a mechanical power to at least one of the front and rear wheels; and
a battery housing configured to removably receive and bold one or more battery packs, wherein the one or more battery packs are configured to power the electric vehicle.

2. The vehicle of claim 1, wherein the controller is in communication with the electric motor.

3. The vehicle of claim 1, further comprising:
a gear coupled to the at least one front wheel or the at least one rear wheel.

4. The vehicle of claim 1, further comprising:
an irradiation control device coupled to at least a portion of the UVC light sources.

5. The vehicle of claim 1, wherein a luminous intensity distribution figure regional area and an irradiation state of the UVC light sources controls a distance of UVC light distribution.

6. The vehicle of claim 1, wherein an irradiation control and irradiating state of the UVC light sources is provided.

7. The vehicle of claim 1, wherein a distance of UVC light within an area of a luminous intensity distribution is controlled.

8. The vehicle of claim 1, wherein a luminous intensity distribution is controlled by dividing the steering element into a plurality of regions.

9. The vehicle of claim 1, wherein the vehicle is a scooter.

10. The vehicle of claim 9, wherein the steering element is a handlebar.

11. The vehicle of claim 9, wherein the handlebar includes first and second distal ends.

12. The vehicle of claim 11, wherein each of the first and second distal ends includes one or more UVC light sources.

13. The vehicle of claim 12, wherein each of the first and second distal ends includes a set of 3 or more UVC light sources.

14. The vehicle of claim 13, wherein each of a set of 3 or more UVC light sources provides illumination in 360 degrees of a selected handlebar section.

15. The vehicle of claim 12, wherein an amount of UVC light emitted that each of the first and second distal ends is 15 mJ/cm2 to 20 mJ/cm2.

16. The vehicle of claim 12, wherein an amount of UVC light emitted that each of the first and second distal ends is mJ/cm2 through 19 mJ/cm21;

17. The vehicle of claim 12, wherein an amount of UVC light emitted that each of the first and second distal ends is 18 mJ/cm2.

18. The vehicle of claim 1, wherein a positioning of the UVC light sources is provided to be incident and cover a surface of the structure that needs to be disinfected

19. The vehicle of claim 1, wherein a shaping of UVC light sources reflectors is configure to be incident and cover a surface of the structure that needs to be disinfected of at least 75% of the surface.

20. The vehicle of claim 1, wherein a shaping of UVC light sources reflectors is configure to be incident and cover a surface of the structure that needs to be disinfected of at least 95% of the surface.
